(19) United States
(12) Patent Application Publication
(10) Pub. No.: US 2007/0084144 A1
(43) Pub. Date: Apr. 19, 2007

(54) Packaging and sterilization of medical devices

(75) Inventors: Roger Labrecque, Londonderry, NH (US); Suzanne Conroy, Dracut, MA (US); Keith M. Faucher, Milford, NH (US); Thomas M. Swanick, Hillsborough, NH (US); Paul Martakos, Pelham, NH (US); Theodore Karwoski, Hollis, NH (US); Steve A. Herweck, Nashua, NH (US); Trevor Carlton, Hudson, NH (US)

Correspondence Address: LAHIVE & COCKFIELD, LLP, ONE POST OFFICE SQUARE, BOSTON, MA, 02109-2127, US

(73) Assignee: ATRIUM MEDICAL CORPORATION, Hudson, NH (US)

(21) Appl. No.: 11/525,328

(22) Filed: Sep. 22, 2006

Related U.S. Application Data
(60) Provisional application No. 60/726,869, filed on Oct. 14, 2005.

(51) Int. Cl. A61L 2/20; A61L 2/07; A61L 2/08; A61L 2/14; B65B 55/02

(52) U.S. Cl. 53/425; 422/28; 422/34; 422/22; 422/26

(57) ABSTRACT

A method for the sterilization and packaging of a chemically sensitive medical device is provided. The chemically sensitive medical device has a coating derived from fish oil, a vitamin E compound or a combination thereof. The packaging pouch for the chemically sensitive medical device comprises a non-permeable chamber and a gas-permeable header. The sterilizing agent is administered to the packaged chemically sensitive medical device at a temperature of between about 20° C. and 40° C.

### RELATED APPLICATIONS

This application claims priority to, and the benefit of, co-pending U.S. Provisional Application No. 60/726869, Oct. 14, 2005, for all subject matter common to both applications. The disclosure of said provisional application is hereby incorporated herein by reference in its entirety.

### FIELD OF THE INVENTION

The present invention relates to packaging and sterilizing implantable medical devices, surgical instruments, or any other medical device in need of sterilization and packaging.

### BACKGROUND OF THE INVENTION

Sterilization of medical devices and packaging of the sterilized medical device is a necessary step in the manufacture and distribution of products in the medical and pharmaceutical industries. Sterilization facilitates aseptic introduction of a device into its intended environment in order to reduce the risk of infection and disease.

Traditionally, sterilization of medical devices has been performed by a variety of techniques well known in the art. Examples of sterilization techniques include use of ethylene oxide (ETO) gas, aqueous glutaraldehyde solution, radiation using gamma or electron-beam radiation, steam and vaporized hydrogen peroxide. While all of these techniques are selectively effective, in some cases, there may be difficulties in using them for certain medical devices which may have chemically sensitive components, such as therapeutic agents, coatings, biomolecular sensors, and the like. For example, steam sterilization is highly effective for sterilization; however, the devices being sterilized by steam must be able to withstand high temperatures and condensation that are a natural byproduct of the steam process. Radiation sterilization, particularly gamma radiation sterilization, can also cause degradation of the sensitive components of a medical device.

### SUMMARY OF THE INVENTION

There is a need for packaging and sterilizing chemically sensitive medical devices. The present invention is directed toward further solutions to address the need for sterilizing medical devices.

In accordance with one aspect of the present invention, a method of packaging and sterilizing a chemically sensitive medical device is provided. Accordingly, the steps of the method include: providing the medical device, wherein the medical device includes a coating derived from a vitamin E compound, fish oil, or a combination thereof; providing a pouch having a non-permeable chamber and a gas-permeable header; placing the medical device in the pouch; sealing the pouch along the gas-permeable header, such that the non-permeable chamber remains accessible through the gas-permeable header; sterilizing the medical device with a sterilizing agent provided through the gas-permeable header to the non-permeable chamber, wherein the sterilizing occurs at a temperature of between about 20° C. and 40° C.; sealing the medical device in the non-permeable chamber within the pouch; and optionally removing the header, leaving the medical device packaged within the non-permeable chamber and sterilized. In one particular embodiment, the sterilizing agent is ethylene oxide (ETO) gas. In one embodiment, the ethylene oxide gas may be administered at about 37° C. In another particular embodiment, the sterilizing agent is gamma radiation. In another embodiment, the sterilizing agent is selected from the group consisting of electron-beam radiation, steam, gas plasma and vaporized hydrogen peroxide.

In accordance with one aspect of the present invention, the method optionally includes the steps of sealing the gas-permeable header; purging the medical device in the pouch with an inert gas; sealing the non-permeable chamber; and optionally removing the gas-permeable header. In accordance with another aspect of the present invention, the method optionally includes the steps of purging the medical device in the pouch with an inert gas; sealing the gas-permeable header; sealing the non-permeable chamber and optionally removing the gas-permeable header. In another embodiment, after the medical device is placed in the pouch, which optionally may or may not be sealed at the gas-permeable header, the method optionally includes the steps of exposing the pouch to vacuum conditions and purging the pouch with an inert gas prior to sealing the non-permeable chamber and/or the gas-permeable header. In yet another embodiment, after the medical device is placed in the pouch, which may or may not be sealed, the method optionally includes the steps of purging the pouch with an inert gas and exposing the pouch to vacuum conditions prior to sealing the non-permeable chamber and/or the gas-permeable header. Accordingly, suitable inert gases include argon and nitrogen.

In accordance with one embodiment of the invention, the combination of packaging and sterilization method can be used to minimize the time that a chemically sensitive medical device is exposed to temperature, humidity, light, oxygen or ambient conditions. By reducing the time that these factors are in effect and packaging the chemically sensitive medical device in a protective environment, the chemistry of the device can be maintained and the shelf life of the product can be extended.

In accordance with one aspect of the present invention, the medical device is an implantable medical device, a surgical instrument, or any other medical device in need of sterilization and/or packaging. In accordance with another aspect of the present invention, the medical device has a coating derived from fish oil, a vitamin E compound or a combination thereof. Accordingly, the fish oil contains a mixture of varying chain length saturated and unsaturated fatty acids, glycerides and triglycerides. In one embodiment, the coating may contain a therapeutic agent. Accordingly, the packaging and sterilization method can minimize degradation of the coating and/or the therapeutic agent and can extend the shelf life of the medical device. In one embodiment, the shelf life is expended for at least about three months.

In accordance with one aspect of the present invention, a desiccant, an oxygen scavenger, an oxygen barrier layer or a combination thereof can be added prior to sealing the pouch. Suitable desiccants include, for example, silica gel, clay, molecular sieves, potassium permanganate, activated carbon and activated alumina. Examples of oxygen scavengers include any inorganic material that can absorb oxygen, for example, iron oxide powders enclosed in sachets, sulfites, bisulfites, butylated hydroxytoluene (BHT), butylated hydroxyanisole (BHA), oxygen absorbable polymers enclosed in a pouch or added inside the packaging (e.g., Chevron-Phillips Chemical Company's ethylene methylacrylate cyclohexene methyl acrylated (EMCM) polymer or Ciba's Specialty Chemical's SHELFPLUS™). Examples of oxygen barriers include, for example, polyvinylidene chloride (PVDC)-coated films and polyvinyl alcohol (PVOH).

### BRIEF DESCRIPTION OF THE DRAWINGS

The aforementioned features and advantages, and other features and aspects of the present invention, will become better understood with regard to the following description and accompanying drawings, wherein:

FIG. 1 is a diagrammatic illustration of a packaging pouch with a non-permeable chamber and a gas-permeable header, in accordance with one aspect of the present invention;

FIG. 2 is a flow chart illustrating a method of packaging and sterilizing a medical device, in accordance with one aspect of the present invention;

FIG. 3 is a flow chart illustrating the method of sterilizing a medical device with vaporized hydrogen peroxide;

FIG. 4 is a flow chart illustrating the method of sterilizing a medical device with ethylene oxide;

FIG. 5 is a comparison of FTIR spectra among an unsterilized coated medical device with coated medical devices that were sterilized with normal ETO and cold ETO;

FIG. 6a and 6b are charts comparing the normalized peak height of the CH₂ band with the cis C═C band and the trans C═C band as analyzed by FTIR, respectively, of a coating comprised of 100% fish oil;

FIG. 7a and 7b are charts comparing the normalized peak height of the CH₂ band with the cis C═C band and the trans C═C band as analyzed by FTIR, respectively, of a coating comprised of 70% fish oil and 30% vitamin E;

FIG. 8a and 8b are charts comparing the normalized peak height of the CH₂ band with the cis C═C band and the trans C═C band as analyzed by FTIR, respectively, of a coating comprised of 30% fish oil and 70% vitamin E; and

FIG. 9 is a comparison of FTIR spectra of an unsterilized and sterilized medical devices coated with 100% fish oil that were sterilized with gamma radiation.

### DETAILED DESCRIPTION

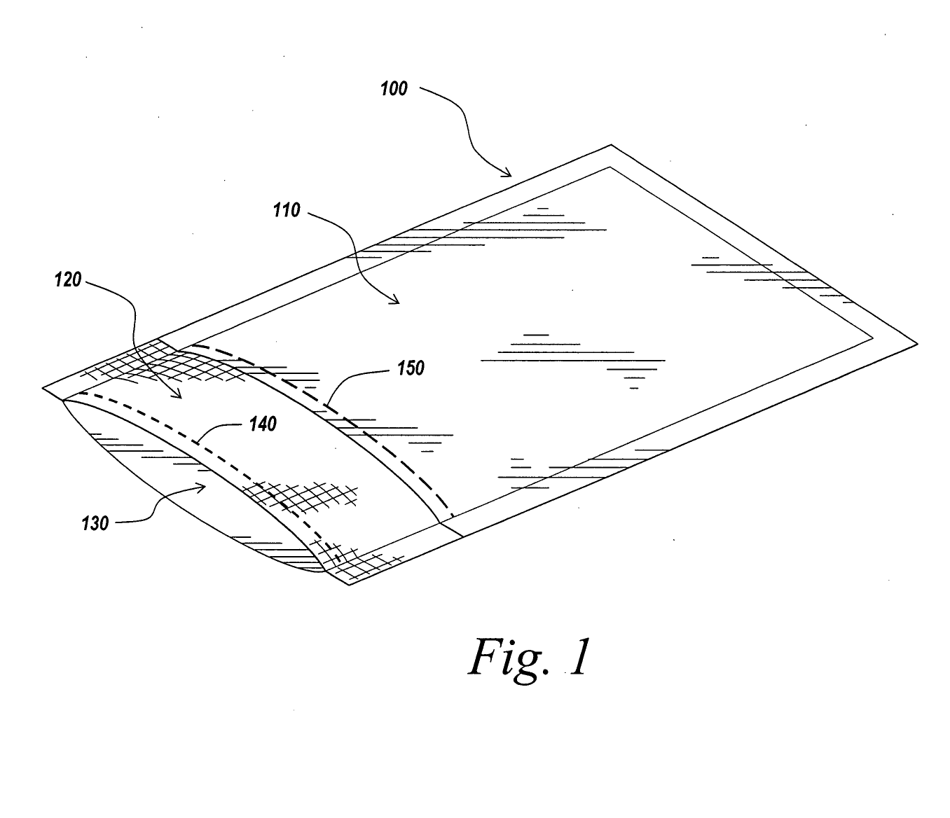

FIGS. 1-9, wherein like parts are designated by like reference numerals throughout, illustrate example embodiments of sterilizing and packaging medical devices according to the present invention. Although the present invention will be described with reference to the example embodiments illustrated in the figures, it should be understood that many alternative forms can embody the present invention. One of ordinary skill in the art will additionally appreciate different ways to alter the parameters of the embodiments disclosed in a manner still in keeping with the spirit and scope of the present invention.

FIG. 1 is a diagrammatic illustration of a packaging pouch with a non-permeable chamber and a gas-permeable header. In accordance with one aspect of the present invention, the packaging pouch 100 is comprised of a non-permeable material 110 and gas-permeable header 120. The permeable material may be composed of a material such as TYVEK®. The material is permeable to the extent of allowing permeation of the material by sterilization gases or products, as described herein. The packaging pouch has an interior chamber 130 capable of containing a variety of medical devices. Appropriate medical devices for use with the present invention include, for example, implantable medical devices (i.e., stents, balloons, catheters, stand alone films, surgical mesh, and the like), surgical instruments (i.e., forceps, scalpels, retractors and the like); and any other medical device or instrument in need of sterilization. In addition, the packaging pouch can be manufactured in any size and/or shape to contain any manner of medical device or instrument.

Referring again to FIG. 1, the packaging pouch has two sites to seal the pouch for sterilization. A first sealing site 140 is located at the opening of the pouch at the top of the gas-permeable header 120. A second sealing site 150 is located at the bottom of the header 120.

One of ordinary skill in the art will appreciate that the packaging pouch 100 is merely one example illustrative embodiment of the packaging structure that can be used in accordance with the present invention. For example, the header 120 can be implemented in a number of different structural embodiments, so long as the functional aspects of the header as described herein, including its permeability to the desired sterilization and inert gases, is maintained. The header 120 can be implemented as a patch, access point, valve, or other gas-permeable implementation that performs in a similar manner as the header described herein with regard to the sterilization methodology of the present invention. The header 120 can be disposed at any location on the packaging that enables the method of sterilization.

Furthermore, the non-permeable material 110 of the packaging can be made of a number of different materials, so long as the functionality of being non-permeable, or substantially non-permeable to air is maintained. For example, plastics, composites, metals, and other materials can provide this functionality.

In addition, one of ordinary skill in the art will appreciate that the present invention is not limited with respect to the location of the seals (the first sealing site 140 and the second sealing site 150), and the specific configuration illustrated and described herein. The seals can be configured and located in a number of different implementations, so long as the seals provide the functionality of sealing off a chamber that includes the gas-permeable area (e.g., header 120) and then subsequently sealing off the non-permeable material 110 of packaging pouch 100 so that the sterilized contents of the packaging pouch 100 is maintained in its sterile environment.

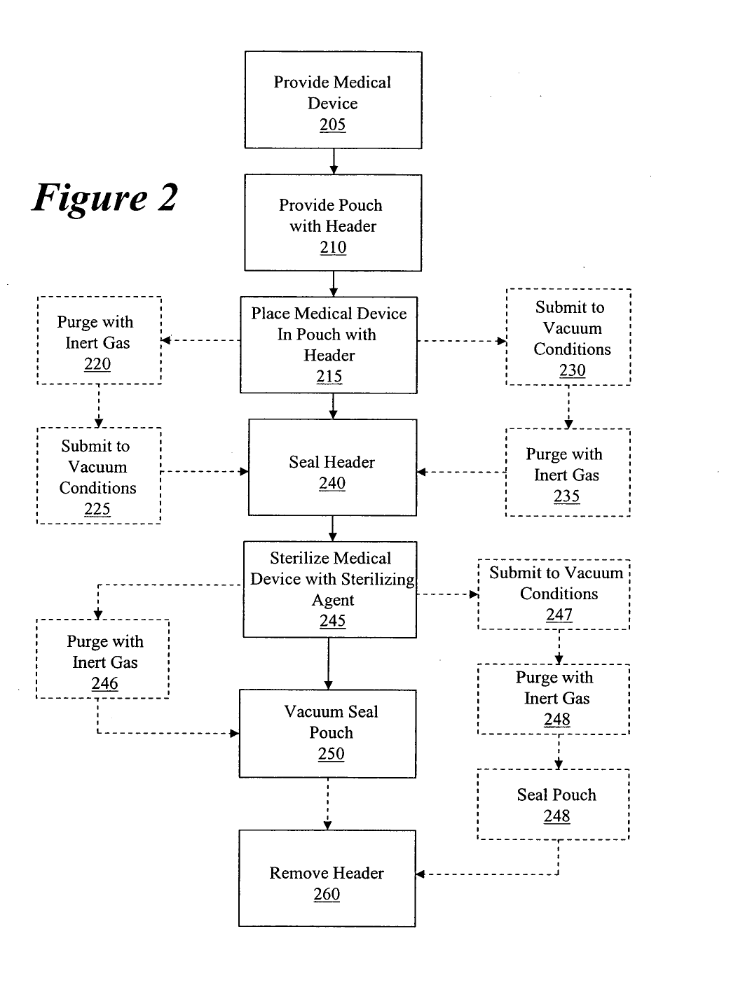

FIG. 2 is flow chart illustrating a method of the present invention, in the form of packaging and sterilizing a chemically sensitive medical device with a sterilizing agent. In accordance with one aspect of the present invention, a chemically sensitive medical device is provided (step 205). As used herein, the term “chemically sensitive” refers to any material that may degrade and/or chemically react upon exposure to heat, steam, water, air or a chemical, or a combination thereof. A chemically sensitive medical device can be a device in which one or more components of the device may degrade and/or chemically react upon exposure to heat, steam, water, air or a chemical or a combination thereof. Chemically sensitive components of the medical device can include, for example, any material that comprises the device itself, as well as any coatings and/or therapeutic agents comprised within the coatings or the medical device.

The coating on the medical device may be chemically sensitive, or may include chemically sensitive components or therapeutics. In accordance with one embodiment of the present invention, the coating can be in the form of a non-polymeric cross-linked gel. It should be noted that the term cross-linked gel, as utilized herein with reference to the present invention, refers to a gel that is non-polymeric and is derived from an oil composition comprising molecules covalently cross-linked into a three-dimensional network by one or more of ester, ether, peroxide, lactone, anhydride and carbon-carbon bonds in a substantially random configuration. In various preferred embodiments, the oil composition comprises a fatty acid molecule, a glyceride, and combinations thereof.

In accordance with one embodiment of the present invention, the medical device includes a coating comprised of a vitamin E compound, fish oil, or a combination thereof. As used herein, the term “vitamin E compound” refers to one or more of alpha-tocopherol, beta-tocopherol, delta-tocopherol, gamma-tocopherol, alpha-tocotrienol, beta-tocotrienol, delta-tocotrienol, gamma-tocotrienol, alpha-tocopherol acetate, beta-tocopherol acetate, gamma-tocopherol acetate, delta-tocopherol acetate, alpha-tocotrienol acetate, beta-tocotrienol acetate, delta-tocotrienol acetate, gamma-tocotrienol acetate, alpha-tocopherol succinate, beta-tocopherol succinate, gamma-tocopherol succinate, delta-tocopherol succinate, alpha-tocotrienol succinate, beta-tocotrienol succinate, delta-tocotrienol succinate, gamma-tocotrienol succinate, mixed tocopherols, vitamin E TPGS, derivatives, analogs and pharmaceutically acceptable salts thereof.

Vitamin E is an antioxidant that is known to slow down autoxidation in fish oil by scavenging free radicals. Due to the large number of sites of unsaturation in fish oil, autoxidation of the fish oil can occur in the presence of heat and/or light, which involves absorption of oxygen into the oil to create hydroperoxides, as shown in Scheme 1.

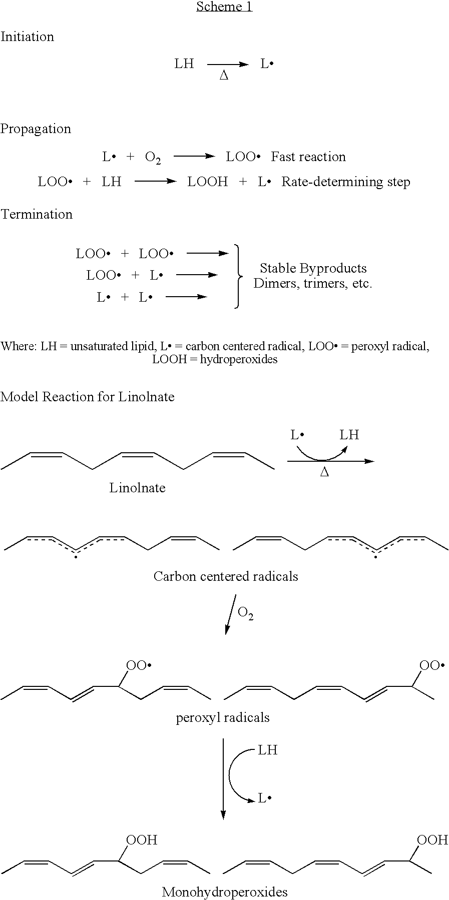

Without being bound by any particular theory, it is thought that the vitamin E competes with unreacted unsaturated lipids in the fish oil during the rate-determining step in the propagation reaction to inhibit autoxidation (Scheme 2). The hydroxyl group of the Vitamin E donates its hydrogen to the lipid peroxyl radical and converts the Vitamin E into a resonance-stabilized radical, as shown in Scheme 2.

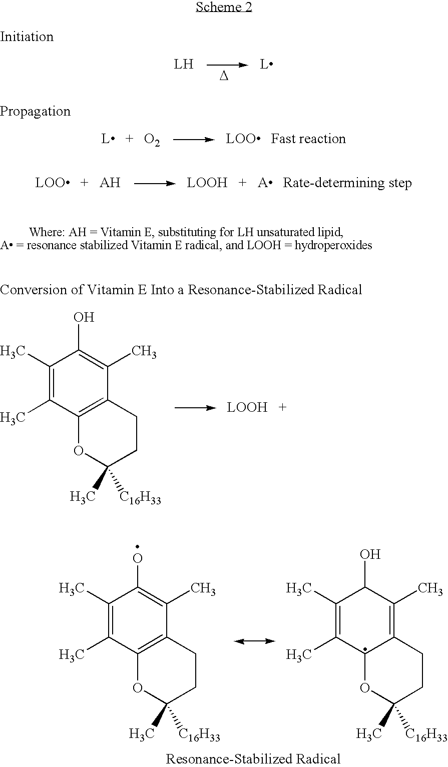

The vitamin E radical can then be further oxidized into alpha-tocopherylquinone (Scheme 3). In-vivo, alpha-tocopherylquinone is reduced into alpha-tocopherylhydroquinone (Scheme 3) and excreted into the feces (Gunstone, F. D. et al. (1995) The Lipid Handbook, Second Edition., Chapman & Hall, New York, NY, pp. 566-571; Gunstone, F. D (1999) Fatty Acid and Lipid Chemistry, Aspen Publications, Gaithersburg, Md., pp. 157-222; Institute of Medicine, (2000) Dietary Reference Intakes for Vitamin C, Vitamin E, Selenium and Carotenoids, National Academy Press, Washington, D.C., Chapter 6, pp. 186-283, the entire contents of each of the foregoing are incorporated herein by reference).

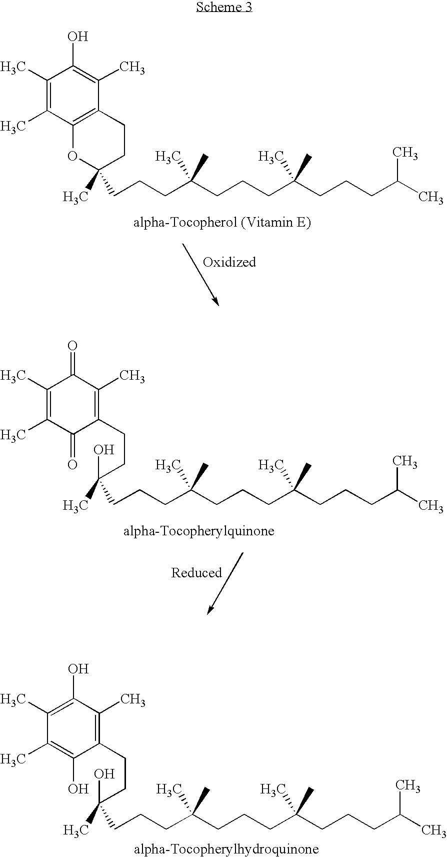

In one embodiment of the present invention, the fish oil of the coating may contain a mixture of varying chain length saturated and unsaturated fatty acids, glycerides, and triglycerides, and may contain one or more fatty acid esters. As used herein, the term “glycerides” refers to mono- and/or diglycerides, which occur naturally in minor amounts of animal and vegetable fats and oils. Monoglycerides are compounds in which one fatty acid chain is covalently bonded to a glycerol molecule through an ester linkage. Diglycerides are compounds in which two fatty acids are covalently bonded to a glycerol molecule through ester linkages. Examples of several monoglycerides [1 (α)-monoglyceride, (1) and 2 (β)-monoglyceride, (2)] and diglycerides [1,2 (α,β)-diglyceride (3) and 1,3 (α,α′)-diglyceride (4)] are shown below:

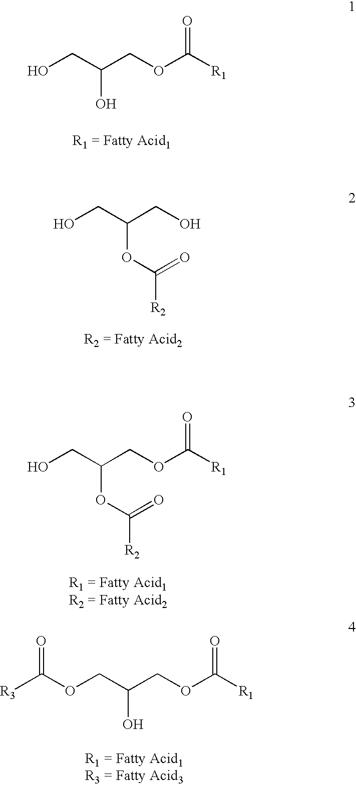

As used herein, the term “triglycerides” generally refers to compounds that consist of three fatty acids attached to one glycerol molecule. If all three fatty acids are identical, it is a simple triglyceride (5). The more common forms, however, are the “mixed” triglycerides (6) in which two or three kinds of fatty acids are present in the molecule.

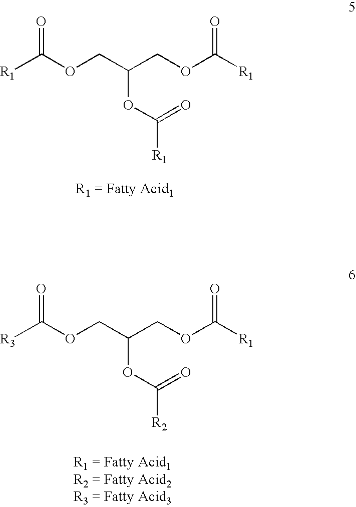

The physical and chemical characteristics of fats and oils are influenced by the kinds and proportions of the fatty acids and in the way in which they are positioned on the glycerol molecule. The predominant fatty acids are saturated and unsaturated carbon chains with an even number of carbon atoms and a single carboxyl group. Fatty acids occurring in fats and oils are classified according to their degree of saturation. As used herein, the term “saturated fatty acids” refers to those fatty acids that contain only single carbon-carbon bonds. Unsaturated fatty acids are those fatty acids that contain one or more carbon-to-carbon double bonds. When the fatty acid contains one double bond it is called “monounsaturated.” If it contains more than one double bond, it is called “polyunsaturated.” Fish oils contain large quantities of a variety of longer chain fatty acids having three or more double bonds, including eicosapentaenoic acid (EPA) and docosahexaenoic acid (DHA).

Omega-3 and omega-6 fatty acids are also important components of some fish oils. The term omega-3 (also labeled as “n-3”, “ω-3”) signifies that the first double bond exists as the third carbon-carbon bond from the terminal methyl end (ω) of the carbon chain. Important omega-3 fatty acids include α-linolenic acid (ALA, 7), eicosapentaenoic acid (EPA, 8), and docosahexaenoic acid (DHA, 9). These three polyunsaturates have either 3, 5 or 6 double bonds in a carbon chain of 18, 20 or 22 carbon atoms, respectively.

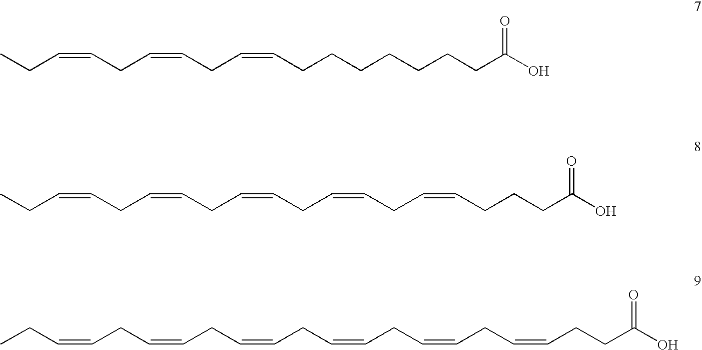

Omega-6 fatty acids are fatty acids where the term “omega-6” signifies that the first double bond in the carbon backbone of the fatty acid, counting from the end opposite the acid group, occurs in the sixth carbon-carbon bond (also labeled as N-6 or ω-6). Important omega-6 fatty acids include linoleic acid (10), γ-linolenic acid (11), eicosadienoic acid (12), di-homo-γ-linolenic acid (13), arachidonic acid (14), docosadienoic acid (15), adrenic acid (16) and docosapentaenoic acid (17).

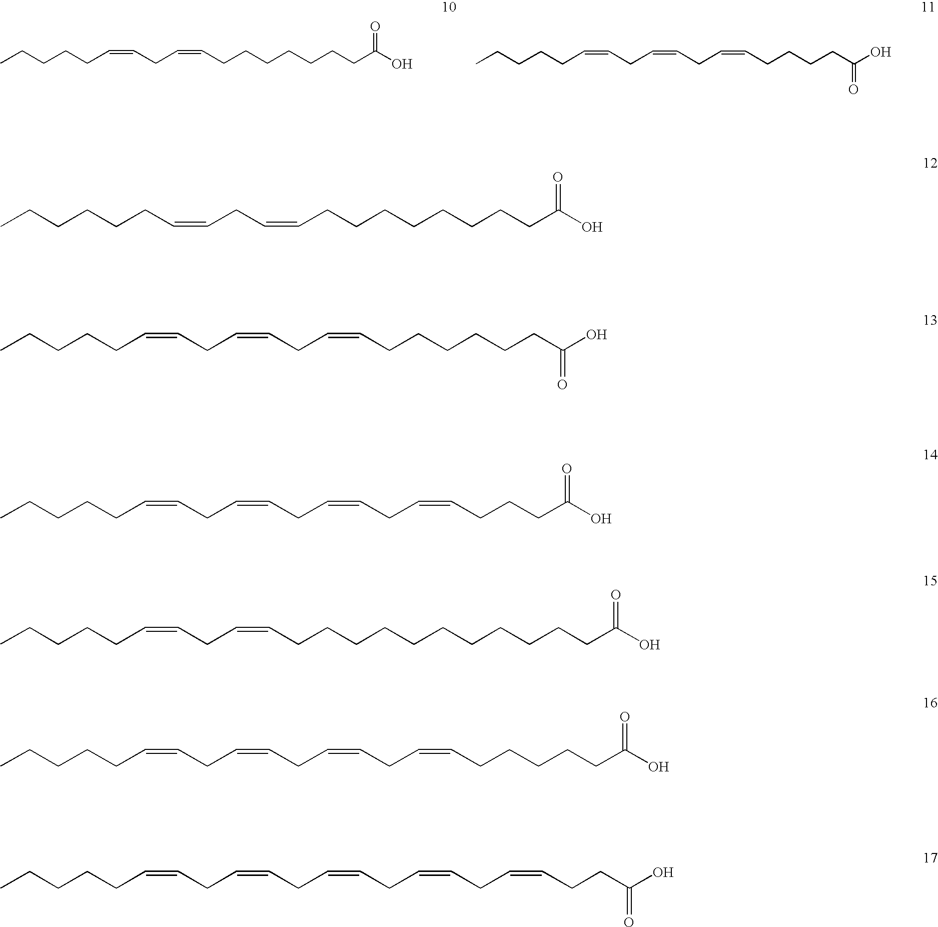

The fish oil of the coating may be comprised of 100% by weight of the trans-isomers of the fatty acids which make up the fish oil. The fish oil of the coating may be comprised of a ratio by weight of mixture of the cis and trans-isomers of the fatty acids which comprise the fish oil. For example, the ratio of the total cis isomers to total trans isomers of the fatty acids of the fish oil may be between about 99:1 to about 0:100. In addition, the fish oil of the coating may contain about 99% by weight of all of the cis-isomers or about 100% of all of the trans-isomers of 4,7,10,13,16,19-docosahexanoic acid, 5,8,11,14,17-eicosapentaenoic acid, 9-tetradecanoic acid (myristoleic acid), palmitoleic acid, 9-hexadecenoic acid, heptadecenoic acid, cis-9-octadecenoic acid (oleic acid), 9,12-octadecadienoic acid (linoleic acid), 9,12,15-octadecatrienoic acid (linolenic acid), 9-eicosenoic acid (gadoleic acid), 11,14-eicosadienoic acid (eicosadienoic acid), 8,11,14-eicosatrienoic acid (dihomo-γ-linolenic acid), 4,7,10,13,16-docosapentaenoic acid, (docosapentaenoic acid).

The ratio of polyunsaturated fatty acids, monounsaturated fatty acids, saturated fatty acids, and omega-3 fatty acids in relationship to each other in the fish oil of the coating is not particularly limited. In one embodiment, the polyunsaturated fatty acids of the fish oil may be comprised of at least about 100% omega-3 fatty acids, or may be comprised of a ratio by weight of between at least about 99:1 to 0:100 omega-3 fatty acids and polyunsaturated fatty acids.

In one embodiment, the coating can contain one or more therapeutic agents. The therapeutic agents suitable for use in the invention are not particularly limited. The therapeutic agents can be hydrophilic, lipophilic, amphiphilic or hydrophobic, and can be dissolved or dispersed in the bio-absorbable carrier, a solvent or the bio-absorbable carrier and the solvent. The therapeutic agent can be any agent having therapeutic value when administered to a subject, for example, a mammal. The therapeutic agent component can take a number of different forms including but not limited to anti-oxidants, anti-inflammatory agents, anti-coagulant agents, drugs to alter lipid metabolism, anti-proliferatives, anti-neoplastics, tissue growth stimulants, analgesics, functional protein/factor delivery agents, anti-infective agents, imaging agents, anesthetic agents, therapeutic agents, tissue absorption enhancers, anti-adhesion agents, anti-migratory agents, pro-healing agents, ECM/Protein production inhibitors, germicides, antiseptics, proteoglycans, GAG's, gene delivery (polynucleotides), polysaccharides (heparin), rapamycin, melatonin, paclitaxel, a protein kinase C inhibitor, cerivastatin, cilostazol, fluvastatin, lovastatin, pravastatin or derivatives, analogs, prodrugs and pharmaceutically acceptable salts thereof, and any additional desired therapeutic agents such as those listed in Table 1 below.

TABLE 1

| CLASS | EXAMPLES |
|---|---|
| Antioxidants | Alpha-tocopherol, lazaroid, probucol, phenolic antioxidant, |
| | resveretrol, AGI-1067, vitamin E |
| Antihypertensive Agents | Diltiazem, nifedipine, verapamil |
| Antiinflammatory Agents | Glucocorticoids (e.g. dexamethazone, |
| | methylprednisolone), leflunomide, NSAIDS, ibuprofen, |
| | acetaminophen, hydrocortizone acetate, hydrocortizone |
| | sodium phosphate, macrophage-targeted bisphosphonates |
| Growth Factor | Angiopeptin, trapidil, suramin |
| Antagonists | |
| Antiplatelet Agents | Aspirin, dipyridamole, ticlopidine, clopidogrel, GP IIb/IIIa |
| | inhibitors, abcximab |
| Anticoagulant Agents | Bivalirudin, heparin (low molecular weight and |
| | unfractionated), wafarin, hirudin, enoxaparin, citrate |
| Thrombolytic Agents | Alteplase, reteplase, streptase, urokinase, TPA, citrate |
| Drugs to Alter Lipid | Fluvastatin, colestipol, lovastatin, atorvastatin, amlopidine |
| Metabolism (e.g. statins) | |
| ACE Inhibitors | Elanapril, fosinopril, cilazapril |
| Antihypertensive Agents | Prazosin, doxazosin |
| Antiproliferatives and | Cyclosporine, cochicine, mitomycin C, sirolimus |
| Antineoplastics | micophenonolic acid, rapamycin, everolimus, tacrolimus, |
| | paclitaxel, QP-2, actinomycin, estradiols, dexamethasone, |
| | methatrexate, cilostazol, prednisone, cyclosporine, |
| | doxorubicin, ranpirnas, troglitzon, valsarten, pemirolast, C- |
| | MYC antisense, angiopeptin, vincristine, PCNA ribozyme, |
| | 2-chloro-deoxyadenosine |
| Tissue growth stimulants | Bone morphogeneic protein, fibroblast growth factor |
| Promotion of hollow | Alcohol, surgical sealant polymers, polyvinyl particles, 2- |
| organ occlusion or | octyl cyanoacrylate, hydrogels, collagen, liposomes |
| thrombosis | |
| Functional Protein/Factor | Insulin, human growth hormone, estradiols, nitric oxide, |
| delivery | endothelial progenitor cell antibodies |
| Second messenger | Protein kinase inhibitors |
| targeting | |
| Angiogenic | Angiopoetin, VEGF |
| Anti-Angiogenic | Endostatin |
| Inhibition of Protein | Halofuginone, prolyl hydroxylase inhibitors, C-proteinase |
| Synthesis/ECM formation | inhibitors |
| Antiinfective Agents | Penicillin, gentamycin, adriamycin, cefazolin, amikacin, |
| | ceftazidime, tobramycin, levofloxacin, silver, copper, |
| | hydroxyapatite, vancomycin, ciprofloxacin, rifampin, |
| | mupirocin, RIP, kanamycin, brominated furonone, algae |
| | byproducts, bacitracin, oxacillin, nafcillin, floxacillin, |
| | clindamycin, cephradin, neomycin, methicillin, |
| | oxytetracycline hydrochloride, Selenium. |
| Gene Delivery | Genes for nitric oxide synthase, human growth hormone, |
| | antisense oligonucleotides |
| Local Tissue perfusion | Alcohol, H₂O, saline, fish oils, vegetable oils, liposomes |
| Nitric oxide Donor | NCX 4016 - nitric oxide donor derivative of aspirin, |
| Derivatives | SNAP |
| Gases | Nitric oxide, compound solutions |
| Imaging Agents | Halogenated xanthenes, diatrizoate meglumine, diatrizoate |
| | sodium |
| Anesthetic Agents | Lidocaine, benzocaine |
| Descaling Agents | Nitric acid, acetic acid, hypochlorite |
| Anti-Fibrotic Agents | Interferon gamma-1b, Interleukin-10 |
| Immunosuppressive/Immunomodulatory | Cyclosporine, rapamycin, mycophenolate motefil, |
| Agents | leflunomide, tacrolimus, tranilast, interferon gamma-1b, |
| | mizoribine |
| Chemotherapeutic Agents | Doxorubicin, paclitaxel, tacrolimus, sirolimus, fludarabine, |
| | ranpirnase |
| Tissue Absorption | Fish oil, squid oil, omega 3 fatty acids, vegetable oils, |
| Enhancers | lipophilic and hydrophilic solutions suitable for enhancing |
| | medication tissue absorption, distribution and permeation |
| Anti-Adhesion Agents | Hyaluronic acid, human plasma derived surgical |
| | sealants, and agents comprised of hyaluronate and |
| | carboxymethylcellulose that are combined with |
| | dimethylaminopropyl, ethylcarbodimide, hydrochloride, |
| | PLA, PLGA |
| Ribonucleases | Ranpirnase |
| Germicides | Betadine, iodine, sliver nitrate, furan derivatives, |
| | nitrofurazone, benzalkonium chloride, benzoic acid, |
| | salicylic acid, hypochlorites, peroxides, thiosulfates, |
| | salicylanilide |
| Antiseptics | Selenium |
| Analgesics | Bupivicaine, naproxen, ibuprofen, acetylsalicylic acid |

Some specific examples of therapeutic agents useful in the anti-restenosis realm include cerivastatin, cilostazol, fluvastatin, lovastatin, paclitaxel, pravastatin, rapamycin, a rapamycin carbohydrate derivative (for example as described in U.S. Patent Application Publication 2004/0235762), a rapamycin derivative (for example as described in U.S. Pat. No. 6,200,985), everolimus, seco-rapamycin, seco-everolimus, and simvastatin.

It should be noted that the sterilization occurs without substantially degrading the chemically sensitive medical device, or any coatings applied to the medical device or any therapeutic agents contained in the coatings. In one embodiment, the sterilization does not substantially alter the coating through the loss of cis double bonds or by altering the degree of cross-linking of the coating.

Referring again to FIG. 2, a packaging pouch 100 is provided (step 210). The medical device is then placed in the packaging pouch (step 215). In accordance with one aspect of the present invention, a desiccant, an oxygen scavenger, an oxygen barrier or a combination thereof may be added to the packaging pouch before the pouch is sealed. Suitable desiccants include, for example, silica gel, clay, molecular sieves, potassium permanganate, activated carbon and activate alumina. Suitable oxygen scavengers include any inorganic material that can absorb oxygen, for example, iron oxide powders, sulfites, bisulfites, butylated hydroxytoluene (BHT), butylated hydroxyanisole (BHA), oxygen absorbable polymers enclosed in a pouch or added inside the packaging (e.g., Chevron-Phillips Chemical Company's ethylene methylacrylate cyclohexene methyl acrylated (EMCM) polymer or Ciba's Specialty Chemical's SHELFPLUS™. Examples of oxygen barriers include, for example, polyvinylidene chloride (PVDC)-coated films and polyvinyl alcohol (PVOH). In one embodiment, the antioxidant and/or the desiccant material may be incorporated into the material that is used to make the pouch. In another embodiment, the packaging pouch may be comprised of an antioxidant packaging material. Examples of antioxidant packaging material include, for example, ethylene methylacrylate cyclohexene methyl acrylated (EMCM) polymer (Chevron-Phillips), Ciba Specialty Chemicals SHELFPLUS™ or other commercially available antioxidant packaging material. In yet another embodiment, the packaging material may optionally be gas-permeable.

In one embodiment, the pouch can be purged with inert gas (step 220), then submitted to vacuum conditions (step 225) before sealing of the gas-permeable header (step 240). In another embodiment, the pouch can be submitted to vacuum conditions (step 230), then purged with an inert gas (step 235) prior to the sealing of the gas-permeable header (step 240). In yet another embodiment, the pouch containing the medical device is sealed at the opening of the pouch at the top of the header (step 240) upon placing the medical device in the pouch. Upon sealing at this point, the packaging pouch is permeable to gasses and vapors in order to allow a sterilization process to occur.

The sealed pouch containing the medical device is then sterilized with a sterilizing agent (step 245). Sterilizing agents are well known in the art and can include, for example, normal ethylene oxide (ETO) gas, cold ETO gas, aqueous glutaraldehyde solution, radiation using gamma or electron-beam radiation and steam, gas plasma, and vaporized hydrogen peroxide (VHP). In one particular embodiment, the sterilizing agent is gamma radiation. In another particular embodiment, the sterilizing agent is cold ETO gas in which the ETO gas is administered at about 20-40° C. In another embodiment, the ETO gas is administered at about 37° C. Accordingly, a medical device with a coating and optionally containing one or more therapeutic agents, upon sterilization with gamma radiation or cold ETO gas, has diminished degradation of the medical device, the coating and/or the one or more therapeutic agents.

Referring back to FIG. 2, after sterilization of the medical device in the packaging pouch, the non-permeable chamber is then vacuum sealed (step 250) at point 150. In accordance with one example embodiment of the present invention, the packaging pouch can be purged with an inert gas, such as argon or nitrogen, prior to vacuum sealing of the non-permeable chamber at the second sealing point 150 (step 246). After the packaging pouch is vacuum sealed at sealing point 150, the header can then be removed (step 260), if desired. In accordance with another embodiment of the present invention, the sterile packaging pouch can be subjected to vacuum conditions (step 247), purged with an inert gas, for example, argon or nitrogen (step 248) and sealed (step 249) at the second sealing point 150. After the packaging pouch is sealed, the header can then be removed (step 260), if desired.

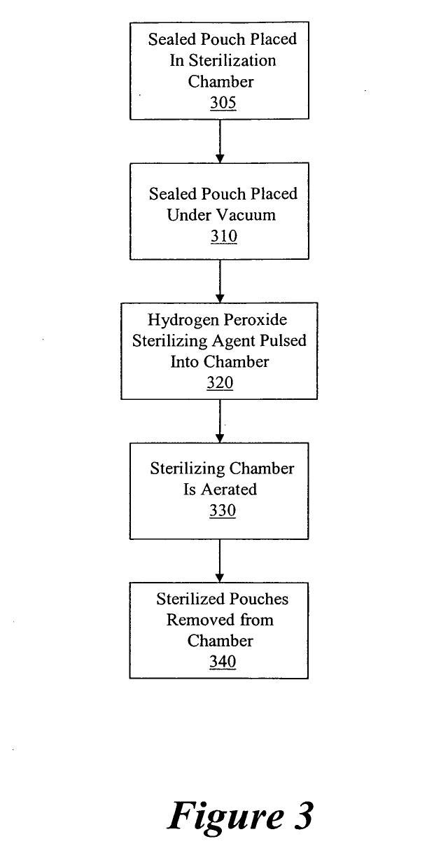

FIG. 3 is a flow chart illustrating the method of sterilizing a medical device with vaporized hydrogen peroxide. Referring to FIG. 3, after sealing the gas-permeable header (step 240, FIG. 2), the sealed pouch containing medical device is placed in the sterilization chamber (step 305), and placed under vacuum (step 310). After the sterilization chamber reaches the appropriate pressure, the hydrogen peroxide sterilizing agent is then injected into the chamber (step 320) with an appropriate weight and number of pulses. After the sterilization is complete, the chamber is aerated for an appropriate amount of cycles (step 330) and the sterilized sealed pouch containing the medical device is removed from the chamber (step 340).

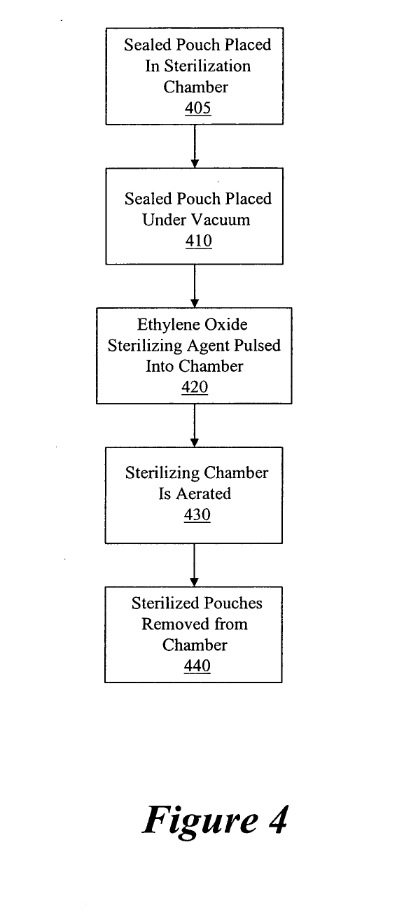

FIG. 4 is a flow chart illustrating the method of sterilizing a medical device with ethylene oxide. Referring to FIG. 4, after sealing the gas-permeable header (step 240, FIG. 2), the sealed pouch containing medical device is placed in the sterilization chamber (step 405), and placed under vacuum (step 410). After the sterilization chamber reaches the appropriate pressure, the ethylene oxide sterilizing agent is then injected into the chamber (step 420) with an appropriate weight and number of pulses. After the sterilization is complete, the chamber is aerated for an appropriate amount of cycles (step 430) and the sterilized sealed pouch containing the medical device is removed from the chamber (step 440).

Various aspects and embodiments of the present invention are further described by way of the following Examples. The Examples are offered by way of illustration and not by way of limitation.

### EXAMPLE # 1

It is known that the chemistry of the coating, predominantly the chemistry of the fish oil, will change over time. These reactions are accelerated in the presence of oxygen, heat and light. The following experiment sought to quantify the effect of different packaging techniques on the aging of the coating. The aging was assessed by FTIR spectroscopy.

A series of 16-month ambient aged coated balloons (70:30 fish oil:vitamin E) were tested for chemical and physical properties. It was found that UV protection is critical to delaying curing of the coating. In this initial experiment, vacuum sealing was slightly better than simply shielding from light or sealing in a foil pouch without Vacuum. It was also found that purging the sample with argon was the best method tested and was superior to purging with nitrogen. The experimental conditions were as follows:

1) Left in Tyvek® and not shielded from light

2) Left in Tyvek® and shielded from light

3) Vacuum sealed in foil pouch

4) Vacuum then purge light with argon and seal in a foil pouch

5) Vacuum then purge heavy with argon and seal in a foil pouch

6) Purge with argon then vacuum seal in a foil pouch

7) Seal in foil pouch (no vacuum or purge)

8) Vacuum then purge heavy with nitrogen and seal in a foil pouch

9) Vacuum then purge light with nitrogen and seal in a foil pouch

10) Purge with nitrogen then vacuum seal in a foil pouch

The IR analysis focused on a few different regions of the spectrum. The first area of interest was the cis and trans bands. After VHP sterilization, the sample lost ˜8.5% of the cis double bonds with no additional formation of trans double bonds. This confirms earlier NMR results that indicated a loss of 5-10% of total double bonds after VHP. After 16 months of ambient temperature aging there were only 3 samples that had a portion of their cis bonds preserved. When a heavy argon purge was used 33% of the cis bonds were retained, when heavy nitrogen purge was used, 14% of the cis bonds were retained (but the sample was orange rather than yellow) and when a light argon purge was used, 2.5% of the cis bonds were retained. The three samples that retained cis bonds also had isolated and conjugated trans bonds where as other unprotected and further oxidized samples showed less and only isolated trans bonds. During oxidation and C═C band isomerization, cis double bonds are converted into isolated and conjugated trans double bonds, but upon further oxidation only isolated trans bonds remain. Thus argon, and to a lesser extent nitrogen, preserve the cis and conjugated trans double bonds in the coating for a longer period of time when compared to the unprotected control. Other samples indicate a retardation of oil auto-oxidation reactions.

An analysis of the carbonyl band region in the FTIR spectrum provides extensive information regarding the oxidative state of aged oils. In unaged fish oil, the carbonyl band is indicative of the ester linkages of the fatty acid chains to the glycerol head group. A broadening of the carbonyl band during aging is indicative of a combination of oxidation and hydrolysis of the fish oil occurring with absorptions from 1730-1680 cm⁻¹ indicating the formation of oxidative byproducts such as ketones, fatty acids in addition to saturated and unsaturated aldehydes. Also, absorptions from 1800-1760 cm⁻¹ indicate the formation of lactone, aliphatic peroxide, and anhydride functional groups created during cross-linking and solidification of the oil. The heavy argon purged sample showed the least amount of carbonyl byproduct formation with little to no cross-linking being observed and with only a small amount of ketone and fatty acid byproduct formation when compared to an unaged control. The light argon and heavy nitrogen purged samples were similar, but they were not as effective as the heavy argon purge as they showed the presence of some cross-linking in addition to increased ketone, fatty acid and aldehyde formation when compared to the heavy argon purged sample, however, the amount of byproducts were ˜40-50% less than the unprotected control. These results indicate that the heavy argon purge packaging is preferred, followed by light argon and/or heavy nitrogen purging.

The standard vacuum seal provided some additional protection over UV protection or straight foil pouching in this initial experiment. The UV protection of the samples was more critical to preserving the samples than vacuum sealing. This was confirmed both physically and chemically.

### EXAMPLE # 2

Exposure to oxygen has been shown to be detrimental to fish oil coating stability and potentially to drug stability, depending on the drug. An example procedure described below measures the residual oxygen in conventional vacuum-sealed packages, in argon-flushed conventional vacuum sealed packages, and in vacuum chamber sealed packages, comparatively. Three pouches containing catheters were sealed by each of the three methods.

Sample “A” is made with a conventional vacuum seal process. Sample “B” is the vacuum then purge with argon process on a conventional vacuum sealer. Sample “C” is the vacuum-chamber sealed packages. Packages from conditions A, B, and C were measured for residual oxygen content with a PBI Dansensor Checkmate. The results are illustrated in Table 2.

TABLE 2

| Sample # | Conditions | Residual Oxygen |
|---|---|---|
| A | Std. vacuum seal package using | 6-10% |
| | conventional vacuum sealer | |
| B | Flush std. package with heavy argon purge | 5.2% |
| | and seal with conventional vacuum sealer | |
| C | Vacuum chamber seal of header pouch; | 1% |
| | flush with argon | |

Vacuum chamber sealing demonstrates nearly an order of magnitude reduction of residual oxygen. Considering the above example, along with the results of analyses of the coating and drug, a vacuum chamber sealer is the preferred packaging method for sensitive coatings.

### EXAMPLE #3

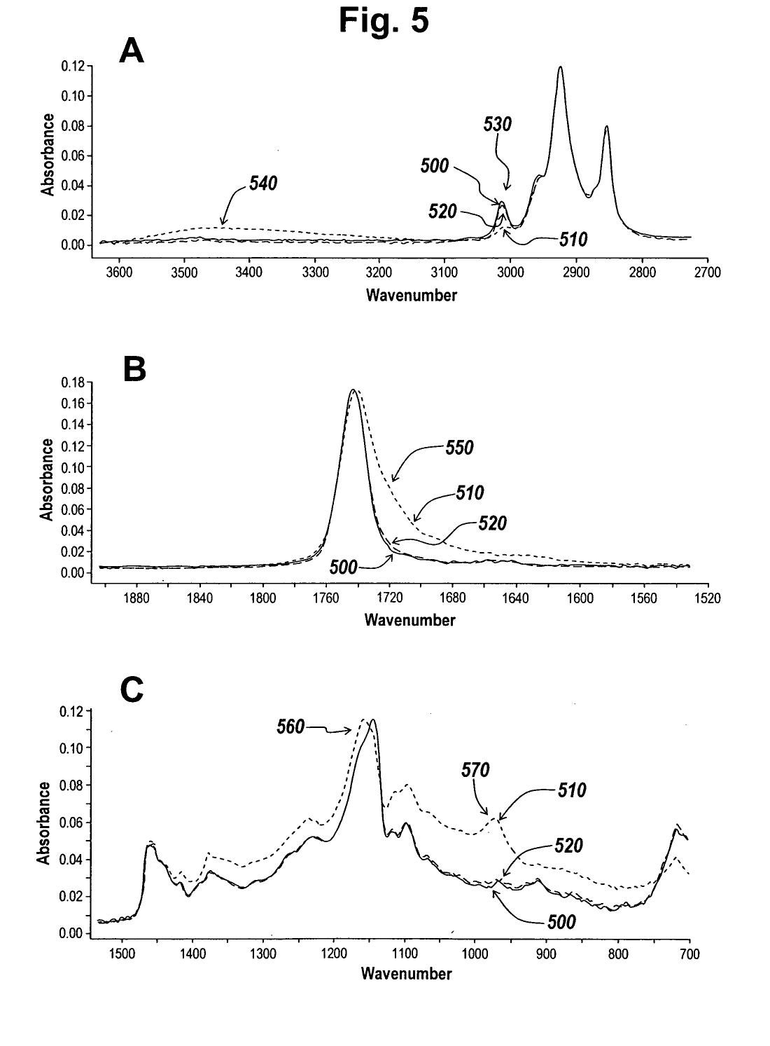

The data in Table 3 show the effects of cold and conventional ETO sterilization on various fish oil/vitamin E formulations when compared to non-sterile controls (500). Conventional ETO sterilization is typically performed at above about 41° C., while cold ETO sterilization may be performed at between about 20-40° C. In general, the FTIR data showed that although cold ETO sterilization (520) does affect the fish oil component of the coating, it is to a much lesser degree than conventional ETO sterilization (510). This is easily illustrated in the FTIR data in FIG. 5 which shows that conventional ETO (510) causes greater oxidation of the fish oil as indicated by increased OH (540), carbonyl (550), and trans C═C (570) band absorptions while decreasing cis C═C (530) band absorption. Cold ETO (520) causes little changes in these absorption bands when compared to normal ETO (510). Also, in general, both data sets support previous observations where an increase in the amount of Vitamin E results in a decrease in the amount of oxidation observed in the fish oil component of the formulation.

TABLE 3

| | | | Conventional ETO | |
|---|---|---|---|---|
| Coating Formulation | Cold ETO Sterilized | | Sterilized | |
| 100% Fish Oil | 1. | Very slight loss of cis C═C | 1. | ˜75% loss of cis C═C |
| | 2. | No detectable increase in | | when compared to non- |
| | | OH band attributed to fatty | | sterile control. |
| | | acid/fatty alcohol/mono- and | 2. | Large OH band intensity |
| | | diglycerides byproducts. | | indicative of formation of |
| | 3. | Slight increase in | | fatty acid/fatty |
| | | aldehyde/ketone/and fatty | | alcohol/mono- and |
| | | acid byproducts. | | diglycerides byproducts. |
| | 4. | No appearance of trans C═C | 3. | Large increase in |
| | | bands. | | aldehyde/ketone/and |
| | 5. | No appreciable | | fatty acid byproducts. |
| | | C—O—C/C—O—O—C | 4. | Strong shifting in |
| | | cross-linking or | | C—O—C/C—O—O—C |
| | | lactone/anhydride/aliphatic | | band indicating peroxide/ |
| | | peroxide band formation. | | ether cross-linking and |
| | | | | slight increase in |
| | | | | lactone/anhydride/aliphatic |
| | | | | peroxide band |
| | | | | formation. |
| | | | 5. | Appearance of isolated |
| | | | | and conjugated trans |
| | | | | C═C bands. |
| 70:30 Fish Oil:Vitamin E | 1. | Little to no change in cis | 1. | Loss of cis C═C. |
| | | C═C | 2. | Increase in OH band |
| | 2. | Slight increase in the OH | | intensity. |
| | | band when compared to non- | 3. | Increase in ketone/fatty |
| | | sterile control, but less than | | acid/aldehyde band. |
| | | normal ETO. | 4. | No appearance of cross- |
| | 3. | Slight increase in | | linking, but appearance |
| | | ketone/fatty acid/aldehyde | | of isolated and |
| | | band when compared to non- | | conjugated trans C═C. |
| | | sterile control, but less than | 5. | NOTE: changes |
| | | normal ETO. | | observed were less than |
| | 4. | No appearance of cross- | | with pure fish oil. |
| | | linking. | | |
| | 5. | Slight trans C═C band | | |
| | | formation. | | |
| 50:50 Fish Oil:Vitamin E | 1. | Slight loss of cis C═C. | 1. | Loss of cis C═C. |
| | 2. | Slight increase in OH band | 2. | Slight increase in OH |
| | | absorption when compared | | band absorption when |
| | | to non-sterile control, which | | compared to non-sterile |
| | | was nearly identical to | | control. |
| | | normal ETO. | 3. | Slight increase in |
| | 3. | Slight Increase in | | ketone/fatty |
| | | ketone/fatty acid/aldehyde | | acid/aldehyde band. |
| | | band when compared to non- | 4. | No appearance of cross- |
| | | sterile control, aldehyde | | linking, but slight |
| | | slightly less than normal | | appearance of trans C═C. |
| | | ETO. | 5. | NOTE: changes |
| | 4. | No appearance of cross- | | observed were less than |
| | | linking or trans C═C band | | with pure fish oil. |
| | | formation. | | |
| 30:70 Fish Oil:Vitamin E | 1. | Little to no detectable | 1. | Slight loss of cis C═C. |
| | | changes observed. | 2. | Slight increase in OH |
| | | | | and ketone/fatty |
| | | | | acid/aldehyde bands. |
| | | | 3. | No appearance of cross- |
| | | | | linking, but slight |
| | | | | appearance of trans C═C. |

| | | |
|---|---|---|
| 100% Vitamin E | No detectable change | No detectable change. |

### EXAMPLE #4

FTIR studies of various rapamycin prodrug formulations (Table 4) also revealed little to no changes in the FTIR spectrum when using cold ETO sterilization when compared to non-sterile controls. There appeared to be little change in the rapamycin prodrug in soluble formulations, but there were slight decreases in the rapamycin prodrug band intensity at 1640 cm⁻¹ for the insoluble formulations. Although there were small changes in OH band intensity for a few of the rapamycin prodrug samples noted in Table 4 after cold ETO sterilization that would be indicative of hydrolysis of the prodrug, it is also likely that these changes could represent hydrolysis and oxidation of the fish oil coating, as that is observed under conventional ETO conditions.

TABLE 4

| Coating Formulation | Cold ETO Sterilized | |
|---|---|---|
| 25% soluble rapamycin | | Little to no change detected, |
| prodrug in 30:70 Fish | | TAFA visible in FTIR |
| oil:Vitamin E | | spectrum. |
| 10% soluble rapamycin | | Little to no change detected, |
| produg in 30:70 Fish | | except for small intensity |
| oil:Vitamin E | | difference in carbonyl band |
| | | (ester). TAFA visible in |
| | | FTIR spectrum. |
| 40% insoluble rapamycin | 1. | Slight loss in cis C═C |
| prodrug in 70:30 Fish | 2. | A very slight increase in the |
| oil:Vitamin E | | OH band. |
| | 3. | Small loss in TAFA FTIR |
| | | band intensity around 1640 cm⁻¹. |
| 30% insoluble rapamycin | 1. | Slight loss in cis C═C |
| prodrug in 85:15 Fish | 2. | Small loss in TAFA FTIR |
| oil:Vitamin E | | band intensity around 1640 cm⁻¹. |

### EXAMPLE #5

Formulations consisting of various combinations of fish oil and vitamin E were coated on a stainless steel substrate and subjected to conventional ETO sterilization and packaging (50° C. ETO sterilization cycle, packaged in TYVEK® pouch) or to the inventive cold ETO process (37° C. ETO sterilization cycle, packaged in a foil header pouch that was purged with Argon then sealed post-sterilization).

The oil was assessed by FTIR before sterilization, after sterilization with both conditions, and for the novel process after 3 months of storage at room temperature. Oxidation of the oil is an indication that the oil is nearing the end of its shelf life. Oxidation of fish oil begins with the isomerization of cis C═C bonds to trans C═C and then proceeds with the subsequent production of numerous oxidative by-products. These complex reactions can be captured by measuring changes in the chemical group spectral absorption band intensity using FTIR.

In order to quantify these changes, the peak height of cis C═C and trans C═C bands were measured and normalized. The cis and trans peak heights are normalized to the peak height of the CH₂ band. Since the CH₂ band remains constant throughout the chemical reactions, we can quantify changes in the relative concentration of the important chemical species by comparing them to the internal standard of the CH₂ band. The measure of the oxidation of the coating, then, is a decrease in the normalized cis C═C peak height and a corresponding increase in the normalized trans C═C peak height.

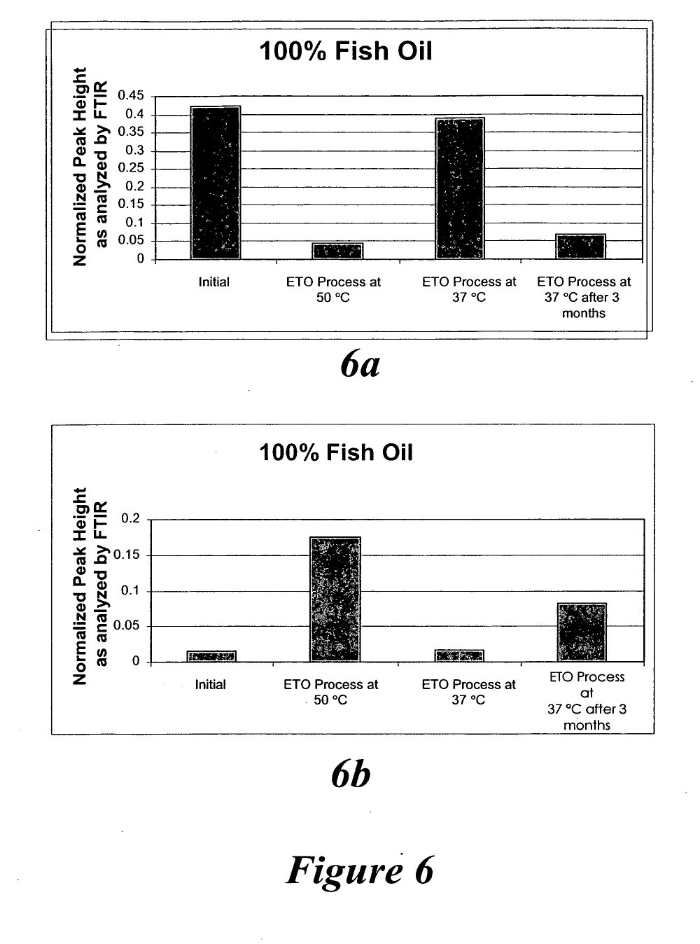

FIGS. 6a and 6b demonstrates that oxidation of the coating comprised of 100% fish oil is a function of sterilization and packaging. In FIG. 6a, it can be seen that immediately after sterilizing at 50° C., the normalized peak height corresponding to the cis C═C bonds significantly decreased, indicating that the coating has lost a considerable amount of the cis C═C bonds due to oxidation. However, sterilization at 37° C. produced only a slight loss in the cis C═C bonds, as evidenced by the small decrease in the normalized peak height. In FIG. 6b, the amount of trans C═C bands increased for ETO sterilization at 50° C., as shown by the increase of the normalized peak height. This data indicated the initiation of the oxidation cascade. On the contrary, sterilization by ETO at 37° C. produced no increase in the normalized peak height of the trans C═C bonds. Both FIGS. 6a and 6b indicate that after 3 months, the cis and trans peak ratios of the coating sterilized at 37° C. began to approach that of the coating immediately after sterilization at 50° C. This indicates an extension in shelf life of at least 3 months for this highly reactive oil.

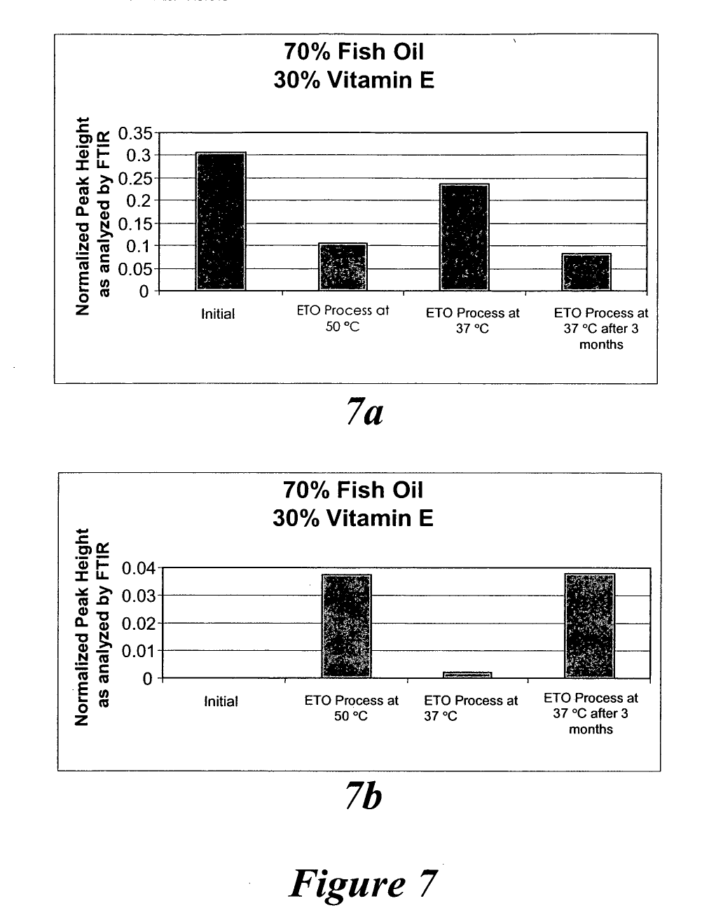

FIGS. 7a and 7b demonstrate that oxidation of the coating comprised of 70% fish oil and 30% vitamin E is also function of sterilization and packaging. In FIG. 7a, it can be seen that immediately after sterilizing at 50° C., the normalized peak height corresponding to the cis C═C bonds decreased by more than half, indicating that the coating has lost a considerable amount of the cis C═C bonds due to oxidation. However, sterilization at 37° C. produced only a small loss in the cis C═C bonds, as evidenced by the small decrease in the normalized peak height. In FIG. 7b, the amount of trans C═C bands increased for ETO sterilization at 50° C., as shown by the increase of the normalized peak height. This data indicated the initiation of the oxidation cascade. On the contrary, sterilization by ETO at 37° C. produced only a small increase in the normalized peak height of the trans C═C bonds. Both FIGS. 7a and 7b indicate that after 3 months, the cis and trans peak ratios of the coating sterilized at 37° C. began to approach that of the coating immediately after sterilization at 50° C. This indicates that there was an extension in shelf life of at least 3 months.

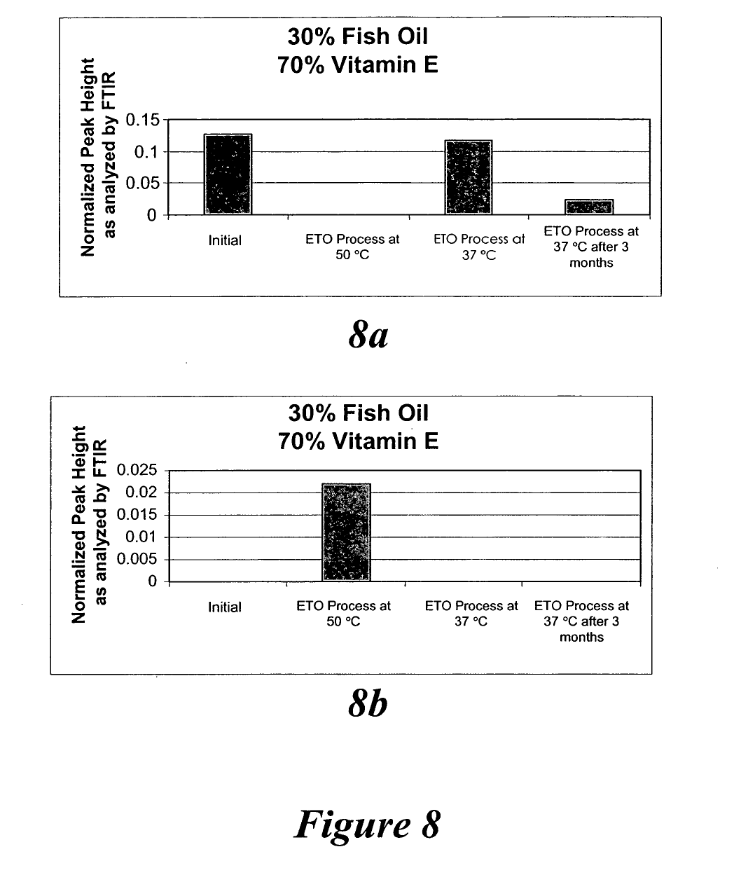

FIGS. 8a and 8b further demonstrate that oxidation of the coating comprised of 30% fish oil and 70% vitamin E is also function of sterilization and packaging. In FIG. 8a, it can be seen that immediately after sterilizing at 50° C., the normalized peak height corresponding to the cis C═C bonds disappeared, indicating that the coating has lost almost all of the cis C═C bonds due to oxidation. However, sterilization at 37° C. produced only a small loss in the cis C═C bonds, as evidenced by the small decrease in the normalized peak height. In FIG. 8b, the amount of trans C═C bands increased for ETO sterilization at 50° C., as shown by the increase of the normalized peak height. This data indicated the initiation of the oxidation cascade. On the contrary, sterilization by ETO at 37° C. produced no increase in the normalized peak height of the trans C═C bonds. Both FIGS. 8a and 8b indicate that after 3 months, the cis and trans peak ratios of the coating sterilized at 37° C. began to approach that of the coating immediately after sterilization at 50° C. This indicates that there was an extension in shelf life of at least 3 months.

The above data show the advantage of including an antioxidant, such as vitamin E, to the coating itself and/or the packaging configuration.

### EXAMPLE #6

Stainless steel stents coated with 100% fish oil, 70:30 and 30:70 fish oil:vitamin E coatings were packaged in the header pouches, sealed, flushed with argon. The header was removed, and the stents were subjected to gamma sterilization. FTIR spectra were taken before packaging and sterilization and after gamma sterilization. Normalized peak ratios, as described in Example #5, were used to compare changes in the cis C═C band intensity by referencing it to the constant symmetric CH₂ peak. Table 5 illustrates the percent decrease of the cis C═C band (as evidenced by the decrease in the normalized peak ratio) after gamma radiation sterilization of the coated stents compared to the non-sterilized coated stents. The slight percent decrease of the normalized value indicated that very little oxidation of the coating had occurred.

TABLE 5

| | Percent decrease of |
|---|---|
| | normalized cis C═C peak ratio |
| | after gamma radiation |
| Formulation | sterilization |
| 100% fish oil | 9.82 |
| 70% fish oil | 10.12 |
| 30% vitamin E | |
| 30% fish oil | 8.82 |
| 70% vitamin E | |

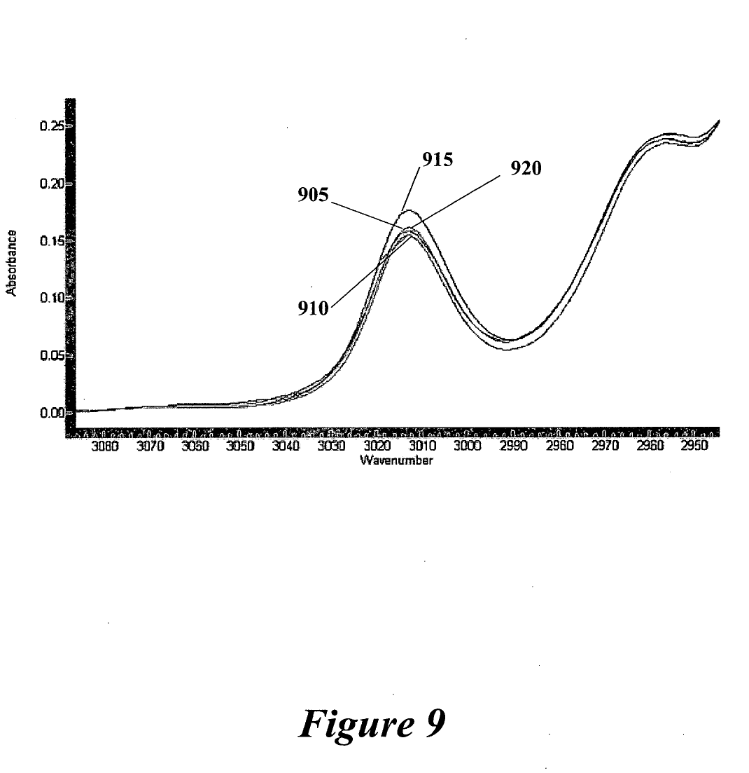

FIG. 9 also demonstrates that oxidation of the coating comprised of 100% fish oil is a function of sterilization by gamma radiation and packaging. FIG. 9 is a comparison of the averaged FTIR spectra at the cis C═C band region of a 100% fish oil coating of a stent before and after gamma radiation sterilization. This comparison shows that there is very little change in the cis C═C band between non-sterilized coating 905, the initially applied non-sterilized coating 910, the gamma sterilized coating immediately after sterilization (915) and gamma sterilized coating 920.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the methods may vary substantially without departing from the spirit of the invention, and exclusive use of all modifications that come within the scope of the appended claims is reserved. It is intended that the present invention be limited only to the extent required by the appended claims and the applicable rules of law.

The section headings used herein are for organizational purposes only and are not to be construed as limiting the subject matter described in any way.

While the present inventions have been described in conjunction with various embodiments and examples, it is not intended that the present teachings be limited to such embodiments or examples. On the contrary, the present inventions encompass various alternatives, modifications, and equivalents, as will be appreciated by those of skill in the art.

The claims should not be read as limited to the described order or elements unless stated to that effect. It should be understood that various changes in form and detail may be made without departing from the scope of the appended claims. Therefore, all embodiments that come within the scope and spirit of the following claims and equivalents thereto are claimed.

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents may be selected for the present invention and embodiments thereof.

What is claimed is:

1. A method of packaging and sterilizing a chemically sensitive medical device, comprising:
providing the medical device, wherein the medical device comprises a coating derived from a vitamin E compound, fish oil, or a combination thereof;
providing a pouch having a non-permeable chamber and a gas-permeable header;
placing the medical device in the pouch;
sealing the pouch along the gas-permeable header, such that the non-permeable chamber remains accessible through the gas-permeable header;
sterilizing the medical device with a sterilizing agent provided through the gas-permeable header to the non-permeable chamber, wherein the sterilizing occurs at a temperature of between about 20° C. and 40° C.;
sealing the medical device in the non-permeable chamber within the pouch; and
optionally removing the header, leaving the medical device packaged within the non-permeable chamber and sterilized.

2. The method of claim 1, wherein the sterilizing agent is ethylene oxide (ETO) gas.

3. The method of claim 1, wherein the temperature is at least about 37° C.

4. The method of claim 1, wherein the sterilizing agent is gamma radiation.

5. The method of claim 1, wherein the sterilizing agent is selected from the group consisting of electron-beam radiation, steam, gas plasma and vaporized hydrogen peroxide.

6. The method of claim 1 wherein the medical device comprises an implantable medical device, a surgical instrument, or any other medical device in need of sterilization.

7. The method of claim 1, wherein the fish oil contains a mixture of varying chain length saturated and unsaturated fatty acids, glycerides and triglycerides.

8. The method of claim 1, wherein the coating further comprises a therapeutic agent.

9. The method of claim 1, wherein the packaging and sterilization method minimizes degradation of the coating.

10. The method of claim 1, wherein the packaging and sterilization method extends the shelf life of the medical device.

11. The method of claim 9, wherein the shelf life of the medical device is extended for about 3 months.

12. The method of claim 1, wherein the combination of packaging and sterilization up to the step of sealing the pouch can be used to minimize exposure time to conditions which are known to degrade the coating or the medical device.

13. The method of claim 12, wherein the packaging reduces exposure to temperature, UV light, humidity, oxygen and ambient conditions.

14. The method of claim 1, wherein after the gas-permeable header is sealed, the method further comprises the step of purging the pouch with an inert gas prior to sealing the non-permeable chamber and removing the gas-permeable header.

15. The method of claim 13, wherein the inert gas is argon or nitrogen.

16. The method of claim 1, wherein after the medical device is placed in the pouch the method further comprises the steps of
exposing the pouch to vacuum conditions; and
purging the pouch with an inert gas prior to sealing the gas-permeable header.

17. The method of claim 16, wherein the inert gas is argon or nitrogen.

18. The method of claim 1, wherein prior to the sealing the gas-permeable header, a desiccant, an oxygen scavenger, an oxygen barrier or a combination thereof is added to the pouch.

19. The method of claim 18, wherein the desiccant is selected from the group consisting of silica gel, clay, molecular sieves, potassium permanganate, activated carbon, activated alumina and a water absorbable polymer.

20. The method of claim 18, wherein the oxygen scavenger is an iron oxide powder, a sulfite, a bisulfite, an oxygen absorbable polymer, butylated hydroxytoluene (BHT), butylated hydroxyanisole (BHA), a commercially available antioxidant packaging or a combination thereof.

21. The method of claim 18, wherein the oxygen barrier is a polyvinylidene chloride (PVDC)-coated film, a polyvinyl alcohol (PVOH) or a combination thereof.

22. The method of claim 1, wherein the pouch is comprised of an antioxidant packaging material.

23. The method of claim 1, wherein the after the medical device is placed in the pouch the method further comprises the steps of
purging the pouch with an inert gas; and
the gas-permeable header is sealed prior to sterilization.

24. The method of claim 23, wherein the inert gas is argon or nitrogen.
